(19) United States
(12) Patent Application Publication
(10) Pub. No.: US 2024/0052319 A1
(43) Pub. Date: Feb. 15, 2024

(54) Methods for Differentiating Epithelial or Basal Cells

(71) Applicant: Lung Biotechnology PBC, Silver Spring, MD (US)

(72) Inventors: Adrian COOPER, Silver Spring, MD (US); Andre DHARMAWAN, Silver Spring, MD (US); Elena FEDERZONI, Silver Spring, MD (US); Megan LEE, Silver Spring, MD (US); Katie WERLE, Silver Spring, MD (US); Kurt WONG, Silver Spring, MD (US)

(21) Appl. No.: 18/354,210

(22) Filed: Jul. 18, 2023

Related U.S. Application Data
(60) Provisional application No. 63/390,454, filed on Jul. 19, 2022.

(51) Int. Cl. C12N 5/071; C12N 5/00

(52) U.S. Cl. CPC C12N 5/0688; C12N 5/0018; C12N 2533/52; C12N 2513/00; C12N 2501/115; C12N 2501/119; C12N 2501/39; C12N 2501/01; C12N 2501/999; C12N 2501/117

(57) ABSTRACT

The present disclosure relates to methods of differentiating pluripotent stem cells or lung progenitor cells into epithelial or basal cells. The present disclosure also relates to epithelial or basal cells made by such methods, organoids containing such epithelial or basal cells, and methods of using the same. The present disclosure also relates to differentiation media for use in the same.

### CROSS REFERENCE TO RELATED APPLICATIONS

This application claims the priority benefit of U.S. Provisional Application No. 63/390,454, filed Jul. 19, 2022, which is incorporated herein by reference in its entirety.

### FIELD OF THE DISCLOSURE

The present disclosure relates to methods of differentiating pluripotent stem cells or lung progenitor cells into epithelial or basal cells. The present disclosure also relates to epithelial or basal cells made by such methods, organoids containing such epithelial or basal cells, and methods of use thereof. The present disclosure also relates to differentiation media for use in the same.

### BACKGROUND

Pluripotent stem cells (PSC) are undifferentiated or partially differentiated cells that can differentiate into various other cell types. Induced pluripotent stem cells (iPSC) are a type of PSC derived from adult somatic cells that have been genetically reprogrammed to an embryonic stem cell (ESC)-like state through the expression of genes and factors important for maintaining the defining properties of ESC. iPSC have generated interest in the medical community recently because they address many obstacles associated with the use of embryonic stem cells, and allow for the generation of patient-specific PSC, which can be genetically corrected, differentiated into adult lineages, and returned to the same patient as an autograft. Yamanaka et al., Cell Stem Cell. 1(1):39-49 (2007); Nishikawa et al., Nat. Rev. Mol. Cell Biol. 9:725 (2008). In addition to genetic disorders, iPSC can be used for tissue regeneration and disease modeling. Kogut et al., Methods Mol. Biol. 1195:1-12 (2014).

### BRIEF SUMMARY

The present disclosure provides methods for differentiating pluripotent stem cells (PSC) or lung progenitor cells (LPC) into epithelial or basal cells.

In some aspects, the method comprises (i) culturing LPC in a base culture medium comprising fibroblast growth factor 2 (FGF2), fibroblast growth factor 10 (FGF10), dexamethasone, cyclic adenosine monophosphate (cAMP), an inhibitor of cyclic nucleotide phosphodiesterases, and a Rho-associated, coiled-coil containing protein kinase (ROCK) inhibitor; (ii) separating the cells of (i) having no or low expression of nerve growth factor receptor (NGFR) to form basal precursor cells; (iii) culturing the cells of (ii) in a base culture medium comprising FGF10, keratinocyte growth factor (KGF), a transforming growth factor β type I receptor kinase (ALK5) inhibitor, an inhibitor of suppressor of mothers against decapentaplegic (SMAD) phosphorylation, and a ROCK inhibitor; and (iv) separating the cells of (iii) having expression of NGFR to form basal cells.

In some aspects, the method further comprises (v) culturing the cells of (iv) having expression of NGFR in a base culture medium comprising FGF10, KGF, an ALK5 inhibitor, an inhibitor of SMAD phosphorylation, and a ROCK inhibitor. In some aspects, the culturing of (v) is on a laminin-coated surface. In some aspects, the laminin is laminin-521.

In some aspects, the culturing of (i) is in a three dimensional (3D) matrix. In some aspects, the culturing of (ii) is in a 3D matrix. In some aspects, the 3D matrix is Matrigel®.

In some aspects, FGF2 is present in the culture medium at a concentration of about 250 ng/mL.

In some aspects, FGF10 is present in the culture medium of (i) at a concentration of about 100 ng/mL. In some aspects, FGF10 is present in the culture medium of (ii) and (iv) at a concentration of about 20 ng/mL.

In some aspects, dexamethasone is present in the culture medium at a concentration of about 50 nM.

In some aspects, cAMP is present in the culture medium at a concentration of about 100 μM.

In some aspects, the inhibitor of cyclic nucleotide phosphodiesterases is 3-isobutyl-1-methylxanthine (IBMX). In some aspects, IBMX is present in the culture medium at a concentration of about 100 μM.

In some aspects, the ROCK inhibitor is Y-27632. In some aspects, Y-27632 is present in the culture medium at a concentration a 10 μM.

In some aspects, KGF is present in the culture medium of (ii) and (iv) at a concentration of about 20 ng/mL.

In some aspects, the ALK5 inhibitor is A83-01. In some aspects, A83-01 is present in the culture medium at a concentration of about 1 μM.

In some aspects, the inhibitor of SMAD phosphorylation is DMH-1. In some aspects, DMH-1 is present in the culture medium at a concentration of about 0.5 μM.

The present disclosure also provides epithelial cells made by a differentiation method disclosed herein, basal cells made by a differentiation method disclosed herein, an organoid containing epithelial cells disclosed herein, an organoid containing basal cells disclosed herein, and certain methods of use thereof.

The present disclosure also provides certain differentiation media. In some aspects, a differentiation medium provided herein comprises a base culture medium, FGF10, KGF, an ALK5 inhibitor, an inhibitor of SMAD phosphorylation, and a ROCK inhibitor. In some aspects, a differentiation medium provided herein comprises a base culture medium, about 20 ng/mL FGF10, about 20 ng/mL KGF, about 1 μM A83-01, about 0.5 μM DMH-1, and about 10 μM Y-27632.

### BRIEF DESCRIPTION OF THE DRAWINGS

Some aspects of the invention are herein described, by way of example only, with reference to the accompanying drawings. With specific reference now to the drawings in detail, it is stressed that the particulars shown are by way of example and for purposes of illustrative discussion of aspects of the invention.

FIG. 1 is a differentiation schematic of the protocol described in Example 1.

FIG. 2A is an exemplary whole-cell image at 4× magnification of cells at day 26 of the differentiation protocol described in Example 1.

FIG. 2B-2C shows flow cytometry plots of cell surface markers EpCAM, NKX2-1, and p63 on day 26 of the differentiation protocol described in Example 1.

FIG. 3A is an exemplary whole-cell image at 4× magnification of cells at day 33 of the differentiation protocol described in Example 1.

FIG. 3B-3C shows flow cytometry plots of cell surface markers EpCAM, NKX2-1, and p63 on day 33 of the differentiation protocol described in Example 1.

FIG. 4A is an exemplary whole-cell image at 4× magnification of cells at day 40 of the differentiation protocol described in Example 1.

FIG. 4B-4E shows a flow cytometry gating strategy to identify EpCAM⁺, NKX2-1⁺, NKX2-1⁻, p63⁺, CK5⁺, and NGFR⁺ cells on day 40 of the differentiation protocol described in Example 1.

FIG. 4F is an exemplary whole-cell image at 10× magnification of cells at day 40 of the differentiation protocol described in Example 1.

FIG. 4G shows a flow cytometry gating strategy to identify CD49f⁺, CK5⁺, CK8⁺, NGFR⁺, and SOX2⁺ cells on day 40 of the differentiation protocol described in Example 1.

FIG. 5A is an exemplary whole-cell image at 4× magnification of cells at day 45 of the differentiation protocol described in Example 1.

FIG. 5B-5E shows a flow cytometry gating strategy to identify EpCAM⁺, NKX2-1⁺, NKX2-1⁻, p63⁺, CK5⁺ and NGFR⁺ cells on day 45 of the differentiation protocol described in Example 1.

FIG. 5F is an exemplary whole-cell image at 10× magnification of cells at day 45 of the differentiation protocol described in Example 1.

FIG. 5G shows a flow cytometry gating strategy to identify CD49f⁺, CK5⁺, CK8⁺, NGFR⁺, and SOX2⁺ cells on day 45 of the differentiation protocol described in Example 1.

FIG. 6A shows a flow cytometry gating strategy to identify EpCAM⁺, NKX2-1⁺, p63⁺, CK5⁺, and NGFR⁺ iPS-BC cells at day 44 before cryopreservation as described in Example 1.

FIG. 6B shows a flow cytometry gating strategy to identify EpCAM⁺, NKX2-1⁺, p63⁺, CK5⁺, and NGFR⁺ control iPS-BC cells at day 47 as described in Example 1.

FIG. 6C shows a flow cytometry gating strategy to identify EpCAM⁺, NKX2-1⁺, p63⁺, CK5⁺, and NGFR⁺ iPS-BC cells after thaw from cryopreservation as described in Example 1.

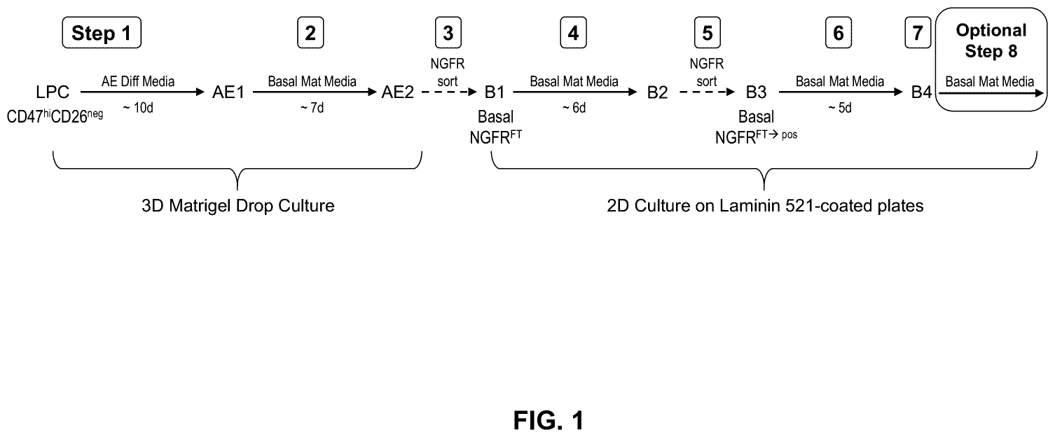

### DETAILED DESCRIPTION

### I. General Definitions

Unless defined otherwise, all technical and scientific terms used herein have the same meaning as commonly understood by one of ordinary skill in the art to which this disclosure belongs. In case of conflict, the present application, including the definitions, will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

### II. Differentiation Methods

The present disclosure relates to methods of differentiating pluripotent stem cells (PSC) or lung progenitor cells (LPC) into epithelial or basal cells. Such methods result, for example, in improved epithelial or basal cell viability, yield, and/or differentiated characteristics.

As used herein, the terms “differentiation” and “differentiating” refer to the process of inducing or reprogramming young or immature cells (e.g., pluripotent stem cells) into more mature or specialized cells (e.g., epithelial or basal cells). In general, differentiation of pluripotent stem cells can be effected, for example, by changing culturing conditions of the cells, such as changing the stimuli agents in a culture medium or the physical state of the cells.

As used herein, the terms “pluripotent stem cell,” “pluripotent stem cells,” and “PSC” refer to young or immature cell(s) that can develop into more mature or specialized cells (e.g., epithelial or basal cells).

In some aspects, PSC include, but are not limited to, embryonic stem cells (ESC), induced pluripotent stem cells (iPSC), embryonic germ cells, adult stem cells, or a combination thereof. In some aspects, the PSC are from a human. In some aspects, the PSC are from an animal. In some aspects, the animal is a sheep, pig or primate.

As used herein, the terms “induced pluripotent stem cell,” “induced pluripotent stem cells,” and “iPSC” refer to cells produced from differentiated adult, neonatal or fetal cells that have been induced or reprogrammed into pluripotent stem cells.

As used herein, the terms “lung progenitor cell,” “lung progenitor cells,” or “LPC” refer to pluripotent cells capable of differentiating into several cell types of the respiratory system, including, but not limited to, pneumocyte type 1 and type II cells, interalveolar cells, smooth muscle cells, alveoli epithelial cells, endothelial cells and erythrocytes. LPC are committed to the pulmonary lineage, retain the ability to self-renew, and can be identified, for example, by cell surface markers or intracellular proteins that include, but are not limited to, transcription termination factor 1 (TTF1), GATA binding protein 6 (GATA6), Est1, Nkx2.1, surfactant protein C (SP-C), forkhead box A1 (FOXA1), FOXA2, SRY-box transcription factor 2 (SOX2), SOX9, CD49f, cytokeratin 8 (CK8), epithelial cellular adhesion molecule (EpCAM), and p63. In some aspects, LPC according to the present disclosure do not express certain markers or exhibit negative or low expression of certain markers, such as markers of endothelial cells (e.g., CD144, CD31), markers of hematopoietic cells (e.g., CD43, CD45, CD235a, or CD41a), and/or markers of pluripotent stem cells (e.g., TRA1-60).

As used herein, the terms “epithelial cell,” “epithelial cells,” or “epithelium” refer to a cell or group of cells that line hollow organs, as well as those that make up glands and the outer surface of the body. Epithelial cells can comprise squamous epithelial cells, columnar epithelial cells, adenomatous epithelial cells, or transitional epithelial cells. Epithelial cells can be arranged in single layers or can be arranged in multiple layers, depending on the organ and location. As used herein, an epithelial cell includes a mature epithelial cell, an epithelial progenitor cell, and an epithelial precursor cell.

In some aspects, cells of the differentiation methods provided herein express or have expression of one or more markers (e.g., cell surface markers, mRNAs, proteins, epigenetic signatures) that typically are associated with epithelial cells such as, for example, T1α, EpCAM, CD49f, and CD104. In some aspects, the cells express or have expression of one or more markers (e.g., cell surface markers, mRNAs, proteins, epigenetic signatures) that typically are associated with basal epithelial cells such as, for example, integrin subunit alpha 6 (ITGA6), integrin subunit beta 4 (ITGB4), keratin 14 (KRT14), KRT15, KRT5 and p63. In some aspects, the cells express or have expression of one or more markers (e.g., cell surface markers, mRNAs, proteins, epigenetic signatures) that typically are associated with differentiated epithelial cells such as, for example, KRT4, KRT6 and KRT8. In some aspects, the cells express or have expression of one or more markers (e.g., cell surface markers, mRNAs, proteins, epigenetic signatures) that typically are associated with airway epithelial cells such as, for example, Hes Related Family BHLH Transcription Factor With YRPW Motif 2 (HEY2), nerve growth factor receptor (NGFR) and bone morphogenetic protein 7 (BMP7). In some aspects, the cells express or have expression of one or more markers (e.g., cell surface markers, mRNAs, proteins, epigenetic signatures) such as, for example, cyclin dependent kinase inhibitor 2B (CDKN2B), Cbp/P300 Interacting Transactivator With Glu/Asp Rich Carboxy-Terminal Domain 2 (CITED2), Cellular Repressor Of E1A Stimulated Genes 1 (CREG1), Inhibitor Of DNA Binding 1 (ID1), Mitogen-Activated Protein Kinase 6 (MAP2K6), Insulin Like Growth Factor Binding Protein 3 (IGFBP3) and IGFBP5.

In some aspects, cells of the differentiation methods provided herein have no or low expression of one or more markers (e.g., cell surface markers, mRNAs, proteins, epigenetic signatures) typically associated with epithelial stem cells such as, for example, nerve growth factor receptor (NGFR), Leucine-rich repeat-containing G-protein coupled receptor 5 (LGR5), or CD24. In some aspects, the cells have no or low expression of one or more markers (e.g., cell surface markers, mRNAs, proteins, epigenetic signatures) typically associated with pluripotent stem cells such as, for example, Lin-28 homologue A (LIN28A), NANOG, POU5F1/OCT4, and SRY-related HMG-box 2 (SOX2). In some aspects, the cells have no or low expression one or more markers (e.g., cell surface markers, mRNAs, proteins, epigenetic signatures) typically associated with terminally differentiated epithelial cells such as, for example, cystic fibrosis transmembrane conductance regulator (CFTR), Forkhead Box J1 (FOXJ1), IVL, keratin 1 (KRT1), KRT10, KRT20, LOR, mucin 1 (MUC1), MUC5AC, secretoglobin family 1A member 1 (SCGB1A1), surfactant protein B (SFTPB) and SFTPD.

In some aspects, cells of the differentiation methods provided herein have no or low expression of NGFR and are basal precursor cells. In some aspects, cells of the differentiation methods provided herein have no or low expression of NGFR and are epithelial or basal cells.

As used herein, “basal cell,” “basal cells” or “BC” are epithelial progenitor cells that reside in the airway epithelium from the trachea and, in diminishing numbers, down to the respiratory bronchioles. They are responsible for normal cell replacement and epithelial remodeling upon lung injury. Basal cells can regenerate the airway epithelium by serving as precursors for essential specialized epithelial cell types, including secretory cells (SC) and multiciliated cells (MCC). Basal cells generally are cells in the deepest layer of stratified epithelium and multilayered epithelium.

In some aspects, basal cells according to the present disclosure possess one or more markers (e.g., cell surface markers, mRNAs, proteins, epigenetic signatures) that include, but are not limited to, CD49f, EpCAM, NGFR, NKX2-1, p63, and CK5. In some aspects, basal cells according to the present disclosure do not express certain markers or exhibit decreased expression of certain markers, such as markers of endothelial cells (e.g., CD144 and/or CD31), markers of hematopoietic cells (e.g., CD43, CD45, CD235a and/or CD41a) or markers of pluripotent stem cells (e.g., TRA1-60).

Methods for generating LPC from PSC (e.g., iPSC) are known and described further herein and, for example, in Jacob et al., Nature Protocols, 14:3303-3332, 2019; Hawkins et al., Cell Stem Cell, 28:79-95, 2021.

In some aspects, the differentiation methods provided herein include certain cell culturing conditions, such as culturing cells in certain culture media.

As used herein, the terms “cell culture,” “cell culturing,” “culture,” “culturing,” and “cultured” refer to the maintenance, growth and/or differentiation of cells in an in vitro environment. The terms “cell culture medium,” “cell culture media,” “culture medium,” and “culture media” refer to a composition for culturing cells that contains nutrients to maintain cell viability, support proliferation and optionally differentiation. A cell culture medium can contain one or more of the following: salt(s), buffer(s), amino acid(s), glucose or other sugar(s), antibiotic(s), serum or serum replacement, and other components such as growth factors, vitamins, etc.

In some aspects, the differentiation methods provided herein refer to a cell culture media as a “base culture medium” supplemented with other components (sometimes referred to herein as “differentiation medium” or “differentiation media”). As used herein, a “base culture medium” or “base culture media” refer to a composition that contains the minimal elements required for maintenance, growth and/or differentiation of cells in an in vitro environment. Examples of a base culture medium include, but are not limited to, Dulbecco's Modified Eagle Medium (DMEM), MEM, Iscove's Modified Dulbecco's Medium (IMDM), Glasgow's modified MEM (GMEM), DMEM/F12, Leibovitz L-15, RPMI-1640, CMRL, Ham F10, and HamF12. In some aspects, a base culture medium is supplemented with one or more other components such as amino acid(s), antibiotic(s), serum, growth factor(s), etc. Such components are well known in the art and described further herein.

In some aspects, the differentiation methods provided herein include certain cell culturing conditions, such passaging cells in certain culture media. As used herein, the terms “passage,” “passaged,” and “passaging” refer to the act of subdividing and plating cells at a lower concentration into one or more cell culture surfaces or vessels, when the cells have proliferated to a desired extent. Passaging typically involves detaching cells by mechanical or enzymatic methods (e.g., incubation in Accutane®) before plating, optionally cells at a certain density. Methods for passaging cells are well known and described further herein.

In some aspects, culturing and passaging in the differentiation methods provided herein are performed with one or more substrates coated onto the cell culture surface or vessel. Such substrates include, but are not limited to, vitronectin, gelatin, laminin (e.g., laminin-111, laminin-211, laminin-121, laminin-221, laminin-332, laminin-311, laminin-321, laminin-411, laminin-421, laminin-511, laminin-213, laminin-521, laminin-423, laminin-522, laminin-523, or a combination thereof), fibronectin, collagen (e.g., collagen I, collagen IV, or a combination thereof), elastin, osteopontin, thrombospondin, mixtures of naturally occurring cell line-produced matrices such as Matrigel™, and synthetic or man-made surfaces such as polyamine monolayers and carboxy-terminated monolayers, or a combination thereof. In some aspects, the substrate is laminin-521. Methods for coating a substrate onto a cell culture surface or vessel are well known and described further herein.

In some aspects, culturing and/or passaging of the differentiation methods provided herein are in two dimensional (2D) cultures. In such cultures, cells are grown as a monolayer on a cell culture surface or vessel, optionally with a substrate coating described above.

In some aspects, culturing and/or passaging of the differentiation methods provided herein are in a three dimensional (3D) matrix. Examples of a 3D matrix include, but are not limited to, polymers (natural or synthetic), ceramics, composites, and a combination thereof. A 3D matrix can be in the form of a hydrogel, a porous 3D scaffold, a rapid-prototyping scaffold, a foam, a sponge, a mesh, microparticles, fiber-like networks, mixtures of naturally occurring cell line-produced matrices such as Matrigel™, and a combination thereof, for example, microparticle-loaded hydrogels.

In some aspects, the differentiation methods provided herein include one or more steps where cultured cells having certain biochemical characteristics are separated. As used herein, the terms “separated” and “separating” refer to a process of isolating one or more specific cell populations from a heterogeneous mixture of cells.

In some aspects, the differentiation methods provided herein include separating cells having no or low expression of NGFR to form basal precursor cells. As used herein, the term “no expression” of NGFR includes, but is not limited to, no detectable expression of NGFR. As used herein, the term “low expression” of NGFR includes, but is not limited to, no detectable expression of NGFR, and NGFR expression that is lower than NGFR expression in a control cell that does not express NGFR and/or is not a basal precursor cell.

In some aspects, the differentiation methods provided herein include separating cells having expression of NGFR to form epithelial or basal cells. As used herein, the terms “expression” or “expresses” with regard to NGFR include, but are not limited to, NGFR expression comparable to mature epithelial or basal cells, or precursors or progenitors thereof, and NGFR expression that is higher than NGFR expression in a control cell that does not express NGFR and/or is not an endothelial or basal cell.

Cell separation methods based on expression of a marker are well known in the art and include, but are not limited to, affinity separation, fluorescence-activated cell sorting (FACS), density gradient centrifugation, immunodensity cell isolation, microfluidic cell sorting, buoyancy-activated cell sorting, aptamer-based cell isolation, complement depletion, and more. Techniques for affinity separation include, but are not limited to, separation using antibody-coated magnetic beads (e.g., immunomagnetic cell separation), affinity chromatography, cytotoxic agents joined to a monoclonal antibody or used in conjunction with a monoclonal antibody, e.g., complement and cytotoxins, and “panning” with an antibody attached to a solid matrix, e.g. plate, or other convenient technique. In some aspects, cells of the present differentiation methods are separated by immunomagnetic cell separation.

Some aspects of the differentiation methods provided herein include culturing cells in a base culture medium containing fibroblast growth factor 2 (FGF2). FGF2, also known as basic fibroblast growth factor or FGF-β, is a growth factor and signaling protein encoded by the FGF2 gene. It possess broad mitogenic and cell survival activities, and is involved in a variety of biological processes, including embryonic development, cell growth, morphogenesis, tissue repair, tumor growth and invasion.

In some aspects, FGF2 is present in the base culture medium at a concentration of from about 100 ng/mL to about 400 ng/mL, or any value or range of values thereof, including, for example, from about 200 ng/mL to about 400 ng/mL, from about 250 ng/mL to about 400 ng/mL, from about 300 ng/mL to about 400 ng/mL, from about 100 ng/mL to about 300 ng/mL, from about 200 ng/mL to about 300 ng/mL, from about 250 ng/mL to about 300 ng/mL, from about 100 ng/mL to about 250 ng/mL, from about 100 ng/mL to about 250 ng/mL, or from about 100 ng/mL to about 200 ng/mL. In some aspects, FGF2 is present in the base culture medium at a concentration of about 100 ng/mL, about 200 ng/mL, about 250 ng/mL, about 300 ng/mL, or about 400 ng/mL. In some aspects, FGF2 is present in the base culture medium at a concentration of about 250 ng/mL.

Some aspects of the differentiation methods provided herein include culturing cells in a base culture medium containing fibroblast growth factor 10 (FGF10). FGF10 is a paracrine signaling molecule seen first in the limb bud and organogenesis development. FGF10 starts the developing of limbs and its involved in the branching of morphogenesis in multiple organs such as the lungs, skin, ear and salivary glands.

In some aspects, FGF10 is present in the base culture medium of (i) at a concentration of from about 25 ng/mL to about 200 ng/mL, or any value or range of values thereof, including, for example, from about 50 ng/mL to about 200 ng/mL, from about 100 ng/mL to about 200 ng/mL, from about 150 ng/mL to about 200 ng/mL, from about 25 ng/mL to about 150 ng/mL, from about 50 ng/mL to about 150 ng/mL, from about 100 ng/mL to about 150 ng/mL, from about 25 ng/mL to about 100 ng/mL, from about 50 ng/mL to about 100 ng/mL, or from about 25 ng/mL to about 50 ng/mL. In some aspects, FGF10 is present in the base culture medium of (i) at a concentration of about 25 ng/mL, about 50 ng/mL, about 100 ng/mL, about 150 ng/mL, or about 200 ng/mL. In some aspects, FGF10 is present in the base culture medium of (i) at a concentration of about 100 ng/mL.

In some aspects, FGF10 is present in the base culture medium of (iii) and/or (iv) at a concentration of from about 5 ng/mL to about 35 ng/mL, or any value or range of values thereof, including, for example, from about 15 ng/mL to about 35 ng/mL, from about 20 ng/mL to about 35 ng/mL, from about 25 ng/mL to about 35 ng/mL, from about 5 ng/mL to about 25 ng/mL, from about 15 ng/mL to about 25 ng/mL, from about 20 ng/mL to about 25 ng/mL, from about 5 ng/mL to about 20 ng/mL, from about 15 ng/mL to about 20 ng/mL, or from about 5 ng/mL to about 15 ng/mL. In some aspects, FGF10 is present in the base culture medium of (iii) and/or (iv) at a concentration of about 5 ng/mL, about 15 ng/mL, about 20 ng/mL, about 25 ng/mL, or about 35 ng/mL. In some aspects, FGF10 is present in the base culture medium of (iii) and/or (iv) at a concentration of about 20 ng/mL.

Some aspects of the differentiation methods provided herein include culturing cells in a base culture medium containing dexamethasone. Dexamethasone is an anti-inflammatory glucocorticoid with a range of effects on cell proliferation and differentiation.

In some aspects, dexamethasone is present in the base culture medium at a concentration of from about 20 nM to about 80 nM, or any value or range of values thereof, including, for example, from about 40 nM to about 80 nM, from about 50 nM to about 80 nM, from about 60 nM to about 80 nM, from about 20 nM to about 60 nM, from about 40 nM to about 60 nM, from about 50 nM to about 60 nM, from about 20 nM to about 50 nM, from about 40 nM to about 50 nM, or about 20 nM to about 40 nM. In some aspects, dexamethasone is present in the base culture medium at a concentration of about 20 nM, about 40 nM, about 50 nM, about 60 nM, or about 80 nM. In some aspects, dexamethasone is present in the base culture medium at a concentration of about 50 nM.

Some aspects of the differentiation methods provided herein include culturing cells in a base culture medium comprising cyclic adenosine monophosphate (cAMP). cAMP facilitates mobilization of glucose and fatty acid reserves and is involved in the function and differentiation of many different cells types.

In some aspects, cAMP is present in the base culture medium at a concentration of from about 25 μM to about 200 μM, or any value or range of values thereof, including, for example, from about 50 μM to about 200 μM, from about 100 μM to about 200 μM, from about 150 μM to about 200 μM, from about 25 μM to about 150 μM, from about 50 μM to about 150 μM, from about 100 μM to about 150 μM, from about 25 μM to about 100 μM, from about 50 μM to about 100 μM, or from about 25 μM to about 50 μM. In some aspects, cAMP is present in the base culture medium at a concentration of about 25 μM, 50 μM, 100 μM, 150 μM, or 200 μM. In some aspects, cAMP is present in the base culture medium at a concentration of about 100 μM.

Some aspects of the differentiation methods provided herein include culturing cells in a base culture medium comprising an inhibitor of cyclic nucleotide phosphodiesterases. Cyclic nucleotide phosphodiesterases are a family of enzymes that hydrolyze the phosphodiester bond in cyclic adenosine monophosphate and cyclic guanosine monophosphate, thereby inhibiting their pulmonary vasodilator properties. In some aspects, the inhibitor of cyclic nucleotide phosphodiesterases is theophylline, 3-isobutyl-1-methylxanthine (IBMX), or Ro 20-1724. In some aspects, the inhibitor of cyclic nucleotide phosphodiesterases is IBMX.

In some aspects, the inhibitor of cyclic nucleotide phosphodiesterases (e.g., IBMX) is present in the base culture medium at a concentration of from about 25 μM to about 200 μM, or any value or range of values thereof, including, for example, from about 50 μM to about 200 μM, from about 100 μM to about 200 μM, from about 150 μM to about 200 μM, from about 25 μM to about 150 μM, from about 50 μM to about 150 μM, from about 100 μM to about 150 μM, from about 25 μM to about 100 μM, from about 50 μM to about 100 μM, or from about 25 μM to about 50 μM. In some aspects, the inhibitor of cyclic nucleotide phosphodiesterases (e.g., IBMX) is present in the base culture medium at a concentration of about 25 μM, 50 μM, 100 μM, 150 μM, or 200 μM. In some aspects, the inhibitor of cyclic nucleotide phosphodiesterases (e.g., IBMX) is present in the base culture medium at a concentration of about 100 μM.

Some aspects of the differentiation methods provided herein include culturing cells in a base culture medium containing a Rho associated kinase (ROCK) inhibitor. ROCK is a serine/threonine kinase that serves downstream effectors of Rho kinases, of which three isoforms exist (RhoA, RhoB and RhoC). A “ROCK inhibitor” can, for example, decrease ROCK expression and/or ROCK activity. Examples of a ROCK inhibitor include, but are not limited to, polynucleotides, polypeptides, and small molecules. More specific examples of a ROCK inhibitor include, but are not limited to, an anti-ROCK antibody, and dominant-negative ROCK variant, siRNA, shRNA, miRNA and antisense nucleic acids that target ROCK. Other examples of a ROCK inhibitor include, but are not limited to, thiazovivin, Y-27632, Fasudil, AR122-86, Y-30141, WF-536, HA-1077, hydroxyl-HA-1077, GSK269962A, SB-772077-B, N-(4-Pyridyl)-N′-(2,4,6-trichlorophenyl)urea, 3-(4-Pyridyl)-1H-indole, (R)-(+)-trans-N-(4-Pyridyl)-4-(1-aminoethyl)-cyclohexanecarboxamide, and ROCK inhibitors disclosed in U.S. Pat. No. 8,044,201, which is herein incorporated by reference in its entirety. In some aspects, the ROCK inhibitor is Y-27632.

In some aspects, the ROCK inhibitor (e.g., Y-27632) is present in the base culture medium at a concentration of from about 1 μM to about 20 μM, or any value or range of values thereof, including, for example, from about 1 μM to about 15 μM, from about 1 μM to about 10 μM, from about 1 μM to about 5 μM, from about 5 μM to about 20 μM, from about 5 μM to about 15 μM, from about 5 μM to about 10 μM, from about 10 μM to about 20 μM, from about 10 μM to about 15 μM, or from about 15 μM to about 20 μM. In some aspects, the ROCK inhibitor (e.g., Y-27632) is present in the base culture medium at a concentration of about 1 μM, about 5 μM, about 10 μM, about 15 μM, or about 20 μM. In some aspects, the ROCK inhibitor (e.g., Y-27632) is present in the base culture medium at a concentration of about 10 μM.

Some aspects of the differentiation methods provided herein include culturing cells in a base culture medium comprising keratinocyte growth factor (KGF). KGF is a member of the heparin-binding fibroblast growth factor family with a distinctive pattern of target-cell specificity. KGF is mitogenically active on epithelial cells and produced by cells of mesenchymal origin.

In some aspects, KGF is present in the base culture medium at a concentration of from about 5 ng/mL to about 35 ng/mL, or any value or range of values thereof, including, for example, from about 15 ng/mL to about 35 ng/mL, from about 20 ng/mL to about 35 ng/mL, from about 25 ng/mL to about 35 ng/mL, from about 5 ng/mL to about 25 ng/mL, from about 15 ng/mL to about 25 ng/mL, from about 20 ng/mL to about 25 ng/mL, from about 5 ng/mL to about 20 ng/mL, from about 15 ng/mL to about 20 ng/mL, or from about 5 ng/mL to about 15 ng/mL. In some aspects, KGF is present in the base culture medium at a concentration of about 5 ng/mL, about 15 ng/mL, about 20 ng/mL, about 25 ng/mL, or about 35 ng/mL. In some aspects, KGF is present in the base culture medium at a concentration of about 20 ng/mL.

Some aspects of the differentiation methods provided herein include culturing cells in a base culture medium comprising an TGF-beta receptor I kinase (ALK5) inhibitor. ALK5 is a serine/threonine protein kinase which forms a heteromeric complex with type II TGF-beta receptors when bound to TGF-beta, transducing the TGF-beta signal from the cell surface to the cytoplasm. This protein is involved in the regulation of many physiological and pathological processes including cell cycle arrest in epithelial and hematopoietic cells, control of mesenchymal cell proliferation and differentiation, wound healing, extracellular matrix production, immunosuppression and carcinogenesis. As used herein, the term “ALK5 inhibitor” refers to a molecule which inhibits or reduces the activity of ALK5, e.g., inhibits or reduces the transduction of the TGF-beta signal from the cell surface to the cytoplasm.

Examples of ALK5 inhibitors include, but are not limited to, A83-01 (Xcess Biosciences (San Diego, Calif.)), ALK5 inhibitor II (Enzo, Farmingdale, N.Y.), ALK5i (Axxora, San Diego, Calif.), SD208 (R & D systems (MN)), TGF-B inhibitor SB431542 (Xcess Biosciences (San Diego, Calif)), ITD-1 (Xcess Biosciences (San Diego, Calif)), LY2109761 (Xcess Biosciences (San Diego, Calif.)), LY2157299 (Xcess Biosciences (San Diego, Calif.)), TGF-β RI Kinase Inhibitor V (EMD Chemicals, Gibstown, N.J.), TGF-beta receptor inhibitor I (EMD Chemicals, Gibstown, N.J.), TGF-β receptor inhibitor IV (EMD Chemicals, Gibstown, N.J.), TGF-beta receptor inhibitor VII (EMD Chemicals, Gibstown, N.J.), TGF-beta receptor inhibitor VIII (EMD Chemicals, Gibstown, N.J.), TGF-beta receptor inhibitor II (EMD Chemicals, Gibstown, N.J.), TGF-beta RI Kinase Inhibitor IV (EMD Chemicals, Gibstown, N.J.), and TGF-beta receptor inhibitor III (EMD Chemicals, Gibstown, N.J.). In some aspects, the ALK5 inhibitor is A83-01.

In some aspects, the ALK5 inhibitor (e.g., A83-01) is present in the base culture medium at a concentration of from about 0.2 μM to about 21.1M, or any value or range of values thereof, including, for example, from about 0.2 μM to about 1.5 μM, from about 0.2 μM to about 11.1M, from about 0.2 μM to about 0.5 μM, from about 0.5 μM to about 2 μM, from about 0.5 μM to about 1.5 μM, from about 0.5 μM to about 11.1M, from about 1 μM to about 21.1M, from about 1 μM to about 1.5 μM, or from about 1.5 μM to about 2 μM. In some aspects, the ALK5 inhibitor (e.g., A83-01) is present in the base culture medium at a concentration of about 0.2 μM, about 0.5 μM, about 1 μM, about 1.5 μM, or about 2 μM. In some aspects, the ALK5 inhibitor (e.g., A83-01) is present in the base culture medium at a concentration of about 1 μM.

Some aspects of the differentiation methods provided herein include culturing cells in a base culture medium comprising an inhibitor of suppressor of mothers against decapentaplegic (SMAD) phosphorylation. SMAD proteins (receptor-regulated or R-SMADs (SMAD 1, 2, 3, 5 and 8) and SMAD4) form a heterocomplex that enters the nucleus where it acts as a transcription factor, inducing the expression of a range of proteins. Phosphorylation of SMAD has strong impact on the structural and functional features of SMAD. Inhibitors of SMAD phosphorylation include, but are not limited to, SB431542, LDN-193189, Noggin PD169316, SB203580, LY364497, A77-01, A83-01, BMP4, GW788388, GW6604, SB-505124, Lerdelimumab, Metelimumab, GC-I008, AP-12009, AP-11014, LY550410, LY580276, LY364497, LY2109761, SB-505124, E-616452 (ALK inhibitor RepSox), SD-208, SMI6, NPC-30345, Ki26894, SB-23580, SD-093, activin-M108A, P144, soluble TBR2-Fc, dolsomorphin dihydrochloride, and derivatives and/or variants thereof. In some aspects, the inhibitor of SMAD phosphorylation is DMI-1-1.

In some aspects, the inhibitor of SMAD phosphorylation (e.g., DMH-1) is present in the base culture medium at a concentration of from about 0.1 μM to about 1.5 μM, or any value or range of values thereof, including, for example, from about 0.2 μM to about 1.5 μM, from about 0.5 μM to about 1.5 μM, from about 1 μM to about 1.5 μM, from about 0.1 μM to about 1 μM, from about 0.2 μM to about 1 μM, from about 0.5 μM to about 1 μM, from about 0.1 μM to about 0.5 μM, from about 0.2 μM to about 0.5 μM, or from about 0.1 μM to about 0.2 μM. In some aspects, the inhibitor of SMAD phosphorylation (e.g., DMH-1) is present in the base culture medium at a concentration of about 0.1 μM, about 0.2 μM, about 0.5 μM, about 1 μM, or about 1.5 μM. In some aspects, the inhibitor of SMAD phosphorylation (e.g., DMH-1) is present in the base culture medium at a concentration of about 0.5 μM.

In some aspects, the differentiation methods provided herein comprise:
(i) culturing LPC in a base culture medium comprising FGF2, FGF10, dexamethasone, cAMP, an inhibitor of cyclic nucleotide phosphodiesterases, and a ROCK inhibitor;
(ii) optionally separating the cells of (i) having no or low expression of NGFR to form basal precursor cells;
(iii) culturing the cells of (ii) in a base culture medium comprising FGF10, KGF, an ALK5 inhibitor, an inhibitor of SMAD phosphorylation, and a ROCK inhibitor; and
(iv) optionally separating the cells of (iii) having expression of NGFR to form epithelial or basal cells.

In some aspects, the differentiation methods provided herein comprise:
(i) culturing LPC in a base culture medium comprising FGF2, FGF10,
dexamethasone, cAMP, an inhibitor of cyclic nucleotide phosphodiesterases, and a ROCK inhibitor;
(ii) separating the cells of (i) having no or low expression of NGFR to form basal precursor cells;
(iii) culturing the cells of (ii) in a base culture medium comprising FGF10, KGF, an ALK5 inhibitor, an inhibitor of SMAD phosphorylation, and a ROCK inhibitor; and
(iv) separating the cells of (iii) having expression of NGFR to form epithelial or basal cells.

In some aspects, the method further comprises:
(v) culturing the cells of (iii) having expression of NGFR in a base culture medium comprising FGF10, KGF, an ALK5 inhibitor, an inhibitor of SMAD phosphorylation, and a ROCK inhibitor.

In some aspects, the culturing of (v) is on a laminin-coated surface. In some aspects, the laminin is laminin-111, laminin-211, laminin-121, laminin-221, laminin-332, laminin-311, laminin-321, laminin-411, laminin-421, laminin-511, laminin-213, laminin-521, laminin-423, laminin-522, laminin-523, or a combination thereof. In some aspects, the laminin is laminin-521.

In some aspects, the culturing of (i) and/or (iii) is in a 3D matrix. In some aspects, the 3D matrix is Matrigel®.

### III. Other Aspects

The present disclosure is also related to epithelial or basal cells made by any of the differentiation methods disclosed herein.

The present disclosure is also related to an organoid comprising epithelial or basal cells made by any of the differentiation methods disclosed herein. As used herein, the term “organoid” refers to a differentiated or partially differentiated, 3D cellular organism derived from PSC (e.g., iPSC) or LPC which is self-organized by densely accumulating cells in a controlled space. Such organisms can be crafted to replicate much of the complexity of an organ, or to express selected aspects of it, for example, only producing certain types of cells.

Methods for maintaining differentiated epithelial or basal cells and organoids are well known and include culturing the cells or organoids in a cell culture medium described herein and/or cryopreservation. Methods for making an organoid typically include culturing cells in a 3D matrix. Examples of a 3D matrix include, but are not limited to, polymers (natural or synthetic), ceramics, composites, or a combination thereof. A 3D matrix can be in the form of a hydrogel, a porous 3D scaffold, a rapid-prototyping scaffold, a foam, a sponge, a mesh, microparticles, fiber-like networks, mixtures of naturally occurring cell line-produced matrices such as Matrigel™, and combinations thereof, for example, microparticle-loaded hydrogels.

The present disclosure is also related to certain methods of using such epithelial and basal cells. In some aspects, the present disclosure provides a method of treating a lung disease comprising administration of epithelial or basal cells or an organoid made by any of the differentiation methods disclosed herein.

The present disclosure is also related to a differentiation medium for differentiating epithelial or basal cells from PSC or LPC.

In some aspects, the differentiation medium comprises a base culture medium, FGF2, FGF10, dexamethasone, cAMP, an inhibitor of cyclic nucleotide phosphodiesterases (e.g., IBMX), and a ROCK inhibitor (e.g., Y-27632). In some aspects, the differentiation medium comprises from about 100 ng/mL to about 400 ng/mL FGF2, from about 25 ng/mL to about 200 ng/mL FGF10, from about 20 nM to about 80 nM dexamethasone, from about 25 μM to about 200 μM cAMP, from about 25 μM to about 200 μM of an inhibitor of cyclic nucleotide phosphodiesterases (e.g., IBMX), and from about 1 μM to about 20 μM of a ROCK inhibitor (e.g., Y-27632). In some aspects, the differentiation medium comprises about 250 ng/mL FGF2, about 100 ng/mL FGF10, about 50 nM dexamethasone, about 100 μM cAMP, about 100 μM of an inhibitor of cyclic nucleotide phosphodiesterases (e.g., IBMX), and about 10 μM of a ROCK inhibitor (e.g., Y-27632).

In some aspects, the differentiation medium comprises a base culture medium, FGF10, KGF, an ALK5 inhibitor (e.g., A83-01), an inhibitor of SMAD phosphorylation (e.g., DMH-1), and a ROCK inhibitor (e.g., Y-27632). In some aspects, the differentiation medium comprises a base culture medium, from about 5 ng/mL to about 35 ng/mL FGF10, from about 5 ng/mL to about 35 ng/mL KGF, from about 0.2 μM to about 2 μM of an ALK5 inhibitor (e.g., A83-01), from about 0.1 μM to about 1.5 μM of an inhibitor of SMAD phosphorylation (e.g., DMH-1), and from about 1 μM to about 20 μM of a ROCK inhibitor (e.g., Y-27632). In some aspects, the differentiation medium comprises a base culture medium, about 20 ng/mL FGF10, about 20 ng/mL KGF, about 1 μM of an ALK5 inhibitor (e.g., A83-01), about 0.5 μM of an inhibitor of SMAD phosphorylation (e.g., DMH-1), and about 10 μM of a ROCK inhibitor (e.g., Y-27632).

### EXAMPLES

Reference is now made to the following examples, which together with the above descriptions illustrate some embodiments of the invention in a non-limiting fashion.

### Example 1

### Differentiation of iPSC to LPC

### Materials and Methods

The following reagents and materials were used:
Iscove's Modified Dulbecco's Medium (IMDM) (Thermo Fisher Scientific; cat #12440053)
Hams F-12 Medium supplemented with L-Glutamine (Corning®; cat #10-080-CV)
GlutaMAX™ Supplement (Thermo Fisher Scientific; cat #35050061)
B-27 supplement (50×) (Thermo Fisher Scientific; cat #17504044)
Bovine serum albumin (BSA); 7.5% in Dulbecco's phosphate buffered saline (DPBS) (Sigma Aldrich; cat #A8412-100ML)
N-2 supplement (Thermo Fisher Scientific; cat #17502048)
L-ascorbic acid (Sigma-Aldrich; cat #A4544-25G)
1-thioglycerol (MTG) (Sigma-Aldrich; cat #M1753-100ML)
Primocin® (InvivoGen; cat #ant-pm-1)
IBMX (3-isobutyl-1-methylxanthine, Sigma-Aldrich; cat #I5879)
8-bromoadenosine 3′,5′-cyclic monophosphate sodium salt (cAMP, Sigma-Aldrich; cat #B7880-100MG)
SAGM™ Small Airway Epithelial Cell Growth Medium BulletKit (Lonza, cat #CC-3118)
Dexamethasone powder (Sigma-Aldrich; cat #D4902-25 MG)
Recombinant human fibroblast grown factor-2 (FGF-2) (Peprotech; cat #AF-100-18B)
Recombinant human keratinocyte growth factor (KGF; FGF-7) (Peprotech; cat #AF-100-19-50UG)
Recombinant human fibroblast growth factor-10 (FGF-10) (Peprotech; cat #AF-100-26-25UG)
DAPT (gamma-secretase inhibitor IX) (Millipore Sigma #565770-10MG)
Dulbecco's modified eagle medium (DMEM)/F12 media (Thermo Fisher Scientific; cat #11320033)
Y-27632 (Rho-associated, coiled-coil containing protein kinase (ROCK) inhibitor) (STEMCELL™ Technologies; cat #72304)
Dispase II (Thermo Fisher Scientific; cat #17105041)
TrypLE™ Express (Thermo Fisher Scientific; cat #12604013)
DMEM, high glucose, GlutaMAX™ Supplement, pyruvate (Thermo Fisher Scientific; cat #10569010)
PneumaCult™ Ex Plus (STEMCELL™ Technologies; cat #05040)
Hydrocortisone Stock Solution (STEMCELL™ Technologies; cat #07926)
Matrigel® GFR Basement Membrane Matrix, LDEV-free (Corning®; cat #354230)
TGF-β RI Kinase Inhibitor IV (A-83-01) (Millipore Sigma; cat #616454)
BMP Inhibitor II, DMH1 (Millipore Sigma; cat #203646)
EZSolution CHIR99021 (GSK-3 inhibitor) (BioVision; cat #1748-5)
CellAdhere Laminin-521 (STEMCELL™ Technologies; cat #200-0117)
STEMdiff™ Definitive Endoderm kit (STEMCELL™ Technologies; cat #05110)

Reagents were prepared as follows:

Ascorbic acid: A solution of 50 mg/mL ascorbic acid was made by dissolving 500 mg of ascorbic acid in tissue culture grade water. The solution was sterile-filtered and stored in 500 μL aliquots at −20° C. for up to 6 months.

MTG: A solution of 13 μL/mL MTG was made by adding 26 μL of MTG to 2 mL IMDM just before making complete serum-free differentiation media, discussed below.

Recombinant human FGF-2 (rhFGF-2): rhFGF-2 was reconstituted to a concentration of 1 mg/mL with sterile water and diluted with 0.1% BSA/phosphate buffered saline (PBS) to a concentration of 250 μg/mL. The solution was stored at −80° C. for up to 3 months.

Recombinant human KGF (rhKGF): rhKGF was reconstituted to a concentration of 0.1 mg/mL with PBS and diluted with 0.1% BSA/PBS to a concentration of 20 μg/mL. The solution was stored at −80° C. for up to 3 months.

Recombinant human FGF-10 (rhFGF-10): rhFGF-10 was reconstituted to a concentration of 0.1 mg/mL with 5 mM sodium phosphate and diluted with 0.1% BSA/PBS to a concentration of 20 μg/mL. The solution was stored at −80° C. for up to 3 months.

DAPT: DAPT was reconstituted to a concentration of 20 mM in sterile dimethyl sulfoxide (DMSO). The solution was aliquoted and stored at −20° C. for up to 3 months.

Y-27632: Y-27632 was reconstituted to a concentration of 10 mM in sterile PBS.

IBMX: IBMX was reconstituted to a concentration of 0.1 M in 200 proof ethanol. The solution was aliquoted and stored at −20° C. for up to one year.

10× cAMP/IBMX: A 10× cAMP/IBMX solution was made by mixing 50 mL cSFDM, 21.5 mg cAMP, and 500 μL of 0.1 M IBMX. The solution was sterile-filtered with a 0.22 μM filter and stored at 4° C. for up to 1 month in the dark.

Dexamethasone: Dexamethasone was reconstituted to a concentration of 1 mM in 63.7 mL of molecular biology grade ethanol. The solution was stored at −20° C. for up to 2 years. A solution of 100 μM dexamethasone was then made by mixing 500 μL of 1 mM dexamethasone and 4.5 mL molecular biology grade ethanol. The solution was aliquoted in 100 μL volumes and stored at −20° C. for up to one year.

Dispase II: Dispase II was dissolved in DMEM medium at 2 mg/mL. The solution was sterile-filtered and stored at 4° C. for up to two weeks or aliquoted and stored at −20° C. for up to 6 months.

A83-01: TGF-β RI Kinase Inhibitor IV (A-83-01) was reconstituted to a concentration of 2 mM in an appropriate volume of DMSO.

DMH1: DMH1 was reconstituted to a concentration of 2 mM in DMSO.

Complete serum-free differentiation media (cSFDM): 375 mL of IMDM, 125 mL of Ham's F-12 media, 5 mL of GlutaMAX™ supplement, 5 mL of B-27 supplement, 3.3 mL of 7.5% BSA, 2.5 mL of N-2 supplement, 500 μL of 50 mg/mL ascorbic acid, 1.5 mL of 13 μL/mL MTG, and 500 μL of Primocin® were mixed and sterile-filtered. The media was stored in the dark at 4° C. for up to 1 month.

Airway differentiation media (ADM): 45 mL of cSFDM base, 5 mL of 10× cAMP/IBMX, 50 μL of 250 μg/mL rhFGF2, 500 μL of 10 μg/mL rhFGF10, 25 μL of 100 μM dexamethasone, and 50 μL of 10 mM Y-27632 were mixed and sterile-filtered. The media was stored in the dark at 4° C. for up to 1 month.

Complete PneumaCult™ Ex Plus Medium: 10 mL PneumaCult™ Ex Plus 50× Supplement, 0.5 mL Hydrocortisone Stock Solution, and 1 mL Primocin® were mixed into 490 mL PneumaCult™ Ex Plus Basal Medium and sterile-filtered.

Complete Proximal Airway Stem Cell Maturation Media (PASC Media): SAGM Media (Lonza), 1 μM A8301, 0.5 μM DHM-1, 20 ng/ml FGF10, 20 ng/ml FGF7 and 10 μM Y-27632 were mixed and sterile-filtered.

Basal Cells Maturation Medium: Complete PneumaCult™ Ex Plus Medium, 1 μM A-83-01, 1 μM DMH1 and 10 μM Y-27632 were mixed and sterile-filtered.

DS/SB Medium (Anteriorization Medium): cSFDM media was supplemented with 2 μM Dorsomorphin and 10 μM SB431542. This was then sterile-filtered.

CBRa media (Lung Progenitor Specification medium): cSFDM media, 3 μM CHIR99021 and 10 ng/mL BMP-4 were mixed and sterile-filtered. Retinoic Acid (RA) was added fresh to make CBRa media the day of media change at a final concentration of 100 nM. The media was stored at 4° C. for up to 1 week.

LPC Sort Buffer: CMF-HBSS, 2% FBS, 25 mM HEPES, 2 mM EDTA, and 10 μM Y-27632 were mixed and sterile-filtered.

An experiment was performed to differentiate lung progenitor cells (LPC) from human induced pluripotent stem cells (iPSC) (BJRiPS iPSC provided by Jintang Du) using the following protocol and reagents.

### Matrigel® Plate Coating Preparation

Matrigel® was thawed overnight on ice and aliquoted into 1.5 mL microfuge tubes according to the dilution factor provided by the manufacturer. Aliquots were stored at −20° C. The day of cell seeding, Matrigel® was diluted with cold DMEM/F-12 media according to the dilution factor provided by the manufacturer so that the volume was sufficient to coat the wells/plates used for cell seeding. Next, the wells of 6-well plates were coated with 1.5 mL Matrigel®/DMEM F-12 solution per well and incubated for at least 1 hour at 37° C. The wells were rinsed with 1 mL of DMEM/F-12 media before cells were added.

iPSC Single Cell Seeding

Cultured BJRiPs (iPSCs) in a 6-well plates were washed with PBS. Cells were detached using 1 mL Accutase® in each well and incubation at 37° C. for 5-7 minutes. The cells were then gently resuspended and pelleted at 200×g for 5 minutes. The iPSC pellet was resuspended in 15 mL of mTeSR1 media with 10 μM Y-27632 to create a single cell suspension with P1000 TIPS. Cells were then counted using a Moxi™ cell counter (ORFLOW® Technology) and seeded onto Matrigel® coated 6-well at 2E6 cells per well. The volume per well was then raised to 3 mL with mTeSR1 media with 10 Y-27632.

### Definitive Endoderm (DE) Differentiation (Day 0)

DMEM/F12 and STEMDiff™ Definitive Endoderm Basal Medium was warmed to 37° C. Supplement MR and Supplement CJ were thawed on ice and diluted 1:100 in STEMdiff™ Endoderm Basal Medium. iPSC wells were washed twice with 1 mL warm DMEM/F12 and then replaced with 2 mL of STEMDiff™ Definitive Endoderm Basal Media containing Supplement MR and Supplement CJ. Cells were then incubated at 37° C. for approximately 24 hours.

### Definitive Endoderm (DE) Differentiation (Day 1-2)

DMEM/F12 and STEMDiff™ Definitive Endoderm Basal Medium was warmed to 37° C. Supplement CJ was thawed on ice and diluted 1:100 in STEMdiff™ Endoderm Basal Medium. Approximately 2 mL of media was added to each well, and cells were incubated at 37° C. for approximately 24 hours. Media was replaced again on Day 2 and the culture continued at 37° C.

### Harvest Cells for Passage and Flow Cytometry QC

Media was removed from 6-well plates and wells were washed twice with 1 mL of PBS. The PBS was removed and 1.5 mL of Accutase™ was added to each well. Plates were incubated for about 4-5 minutes at 37° C. or until the cells easily detached. Harvested cells were diluted with an equal volume of DMEM/F12 media. Cells were pipetted up and down with a p1000 pipettor to dissociate cell clumps into single cells.

Cells were then filtered with a 30 μM filter to remove debris and spun at 200 g for 5 minutes. The media was aspirated and approximately 5 mL FACS Wash or DMEM/F12 was added. Cells were pipetted up and down with a p1000 pipettor to dissociate cell clumps into single cells, and counted with a Moxi™ cell counter (ORFLOW® Technology).

Approximately 250,000 to 300,000 cells were set aside for flow cytometry quality control (QC) assays. The remaining cells were spun down and resuspended to a concentration of approximately 1 million cells per mL in DMEM/F12 media. Cells were seeded into Matrigel®-coated 6-well plates at approximately 208,000 cells/cm 2.

### Anterior Foregut Endoderm (AFE) Differentiation (Day 3)

Following incubation, media was aspirated and 2 mL of AFE DS/SB media supplemented with 2 mL Y-27632 was added to each well. Plates were incubated at 37° C. for 24 hours. For the first 24-48 hours of cell seeding, Y-27632 was added to the media (10 μM final concentration). Media was changed in the same manner every 24 hours until day 6.

Spent medium was aspirated from cells, and cells gently washed twice with approximately 1 mL DMEM/F12 media. 2 mL of CBRa media was added to well and cells were incubated at 37° C. for 48 hours. Media was replaced again on Days 8, 10, 12 and 14.

### Example 2

### Differentiation of LPC to Basal Cells

The following reagents and materials were used:
Fluorescein isothiocyanate (FITC) anti-human CD326 (Ep-CAM) (BioLegend; Cat #324204)
PE anti-human CD26 (BioLegend; cat #302706)
BV421 anti-human CD47 (BioLegend; cat #323116)
FITC Mouse IgG2b, k isotype control (BioLegend; cat #400310)
PE Mouse IgG2a, k isotype control (BioLegend; cat #400212)
BV421 Mouse IgG1, k isotype control (BioLegend; cat #400158)
Matrigel® GFR Basement Membrane Matrix, LDEV-free (Corning®; cat #354230)
UltraComp eBeads™ Compensation beads (Thermo Fisher; cat #01-2222-41)
Ghost Dye™ Red780 (Tonbo Biosciences; cat #13-0865-T100)

The following reagents were prepared:

Sort Wash Buffer: CMF-PBS, 1% FBS, 1 mM EDTA, and 25 mM HEPES were

mixed and sterile-filtered.

LPC Sort Buffer: CMF-HBSS, 2% FBS, 2 mM EDTA, 25 mM HEPES, and 10 μM Y-27632 were mixed and sterile-filtered.

### EPCAM/CD47/CD26 Surface Staining:

Cells were harvested according to the protocol disclosed in Example 1. Cells were counted and prepared for staining. Approximately 150,000-200,000 cells were stained for FITC anti-human CD-326 as a single stain control, a drop of UltraComp eBeads™ Compensation beads for phycoerythrin (PE) single stain control, BV421 anti-human CD47 single stain control, Ghost Dye™ Red780 single stain control, and isotype controls (FITC, PE, and/or BV421) with Ghost Dye™ Red780. Approximately 14E6 cells were stained for EpCAM, CD47 and CD26 with Ghost Dye™ Red780. Cells were washed with PBS after counting and centrifuged at 200×g for 5 minutes at 8° C. Simultaneously, Ghost Dye™ Red780 was diluted 1:750-1:1000 dilution with PBS. Supernatant was aspirated and 100 μL Ghost Dye™ Red780 was added and incubated at room temperature for 10 minutes. After incubation, the cells were washed once with 1 mL Sort Wash Buffer and then centrifuged at 200×g for 5 minutes at 8° C.

In the appropriate samples, the following was added and cells were stained for 30 minutes on ice or at 4° C.:
0.8 μL FITC anti-human CD-326 single stain control per 100 μL LPC Sort Buffer (for up to 1E6 cells)
2.5 μL BV421 anti-human CD47 single stain control per 100 μL LPC Sort Buffer (for up to 1E6 cells)
0.2 μL FITC Mouse IgG2b, k isotype control+0.6 μL PE Mouse IgG2a, k isotype control+1.2 μL BV421 Mouse IgG1, k isotype control per 100 μL LPC Sort Buffer (for up to 1.5E6 cells)
2.5 μL BV421 anti-human CD47+0.8 μL FITC anti-human CD-326+2.5 μL PE anti-human CD26 per 100 μL LPC Sort Buffer (for up to 1.5E6 cells)

Supernatant was then aspirated and LPC Sort buffer was added in the samples so that the final concentration is approximately 1E6-3E6 cells/mL. The cells were filtered using the flow tube with 40 μM filter to remove clumps from the cell suspension before running cells on the BD FACSAria™ Fusion.

CD47hi/CD27—Sorting with BD FACSAria™ Fusion:

The parameters used were as follows: FSC: 260; SSC: 270; AF488: 350; PE: 360; APC-Cy7: 440; BV421: 275; and FSC Scaling Area: 0.3.

All the compensation single color control were ran and the compensation matrix was calculated before running the samples. All isotype control and LPC samples were ran in the BD FACSAria™ Fusion with the appropriate compensation matrix. A collection tube with 5-6 mL of LPC Sort buffer was then made and cells were sorted based on single cells gates (FSC-H vs. FSC-w and SSC-H vs. SSC-W); live cells (Ghost Dye Red780 low) in APC-Cy7 channel; EpCAM+ in AF488 channel; and CD47hi/CD26− in PE/BV421 channel. Cells were also sorted with purity mask for 480,000 cells per 12-well plate (3D) for each sample.

Replating cells in 3D Matrigel®

After cells were collected, they were centrifuged at 200×g for 5 minutes at 8° C. The media was aspirated and undiluted Matrigel® GFR was added on ice to reach a concentration of approximately 600 cells/μL. Approximately 45-50 μL droplets were placed and spread on the bottom of each well of a 12-well plate on ice. The drops were then incubated at 37° C. for 15-20 minutes, 1 mL of ADM was added to each well and then incubated at 37° C. for 48-72 hours. Media was not replaced on Day 16 of differentiation.

### Differentiation Day 17-Day 24 (LPC to Basal Cell (BS) Differentiation Day 1-Day 9)

Media was aspirated from each well and 1 mL of ADM was added. Cells were then incubated at 37° C. for 48-72 hours. Media was replaced every 24-72 hours until differentiation Day 26.

### Differentiation Day 26 (LPC to BS Differentiation Day 10)

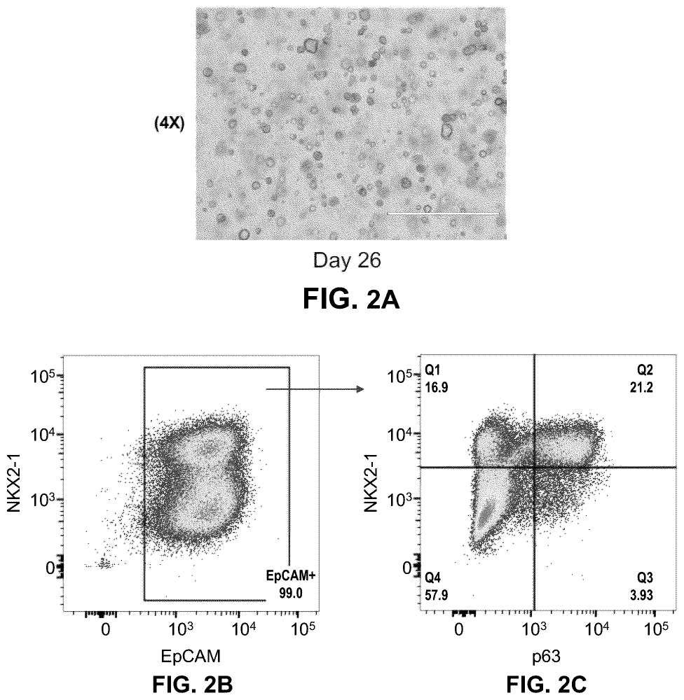

Media was aspirated and 2 mg/mL Dispase was added to cover the droplet (typically 1 mL/well) and then incubated at 37° C. for 60 minutes until the Matrigel® was fully dissolved. Cells were gently pipetted 3-5 times. Dissociated organoids were transferred using a p1000 pipette to a new 15-mL conical tube and an equivalent volume of DMEM was added. Spheres were centrifuged at 300×g for 2 minutes at 4° C. Supernatant was aspirated and 1 mL TrypLE™ per dissociated drop was added. TrypLE™ and cells were transferred a well of a 12-well plate and incubated for 10-12 minutes at 37° C. Cells were incubated until they were entirely dissociated. An equivalent volume of DMEM/F12+10% FBS was added to each well. Dissociated cells were then collected, filtered using a 30 μM filter to remove debris and counted on a Moxi™ cell counter (ORFLOW® Technology). Approximately 100,000 cells were used for QC and remaining cells were replated in 3D Matrigel®. An exemplary image of whole cells at Day 26 differentiation is shown in FIG. 2A. QC plot of cell surface markers EpCAM, NKX2-1 and p63 from day 26 are shown in FIG. 2B-2C. Approximately 1 mL of media was replaced on differentiation Day 28 and Day 30 for all wells and cells remained at 37° C. for 24-48 hours.

Harvesting AE2 from 3D Matrigel® Culture for NGFR Sort and QC (Day 31-Day 40)

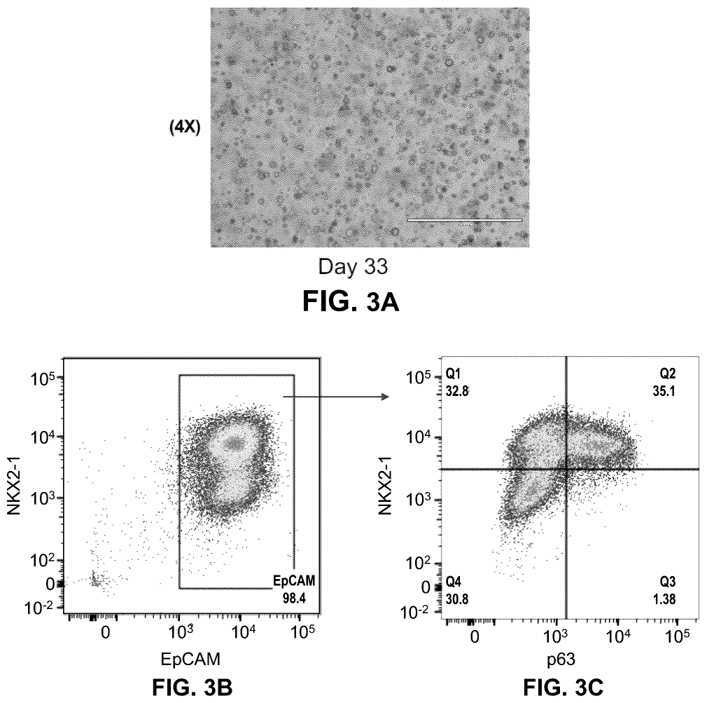

Cells were harvested according to protocol described in Example 1 and sorted using Magnetic labeling and Magnetic separation for NGFR according to manufacturer's protocol. Before seeding, Laminin-521-coated plates were prepared by incubating a solution of 5 μg/mL Laminin-521 in each well of 6-well plate for either 2 hours at 37° C. or overnight at 4° C. After sorting, NGFR positive cells were seeded on Laminin-521 plates at a cell density of approximately 5,000 cells/cm 2 cells and incubated for 24 hours at 37° C. An exemplary image of cells at Day 33 of differentiation is shown in FIG. 3A (4× magnification) and FIG. 3F (10× magnification). QC plots of cell surface markers EpCAM, NKX2-1 and p63 from Day 33 are shown in FIG. 3B-3C.

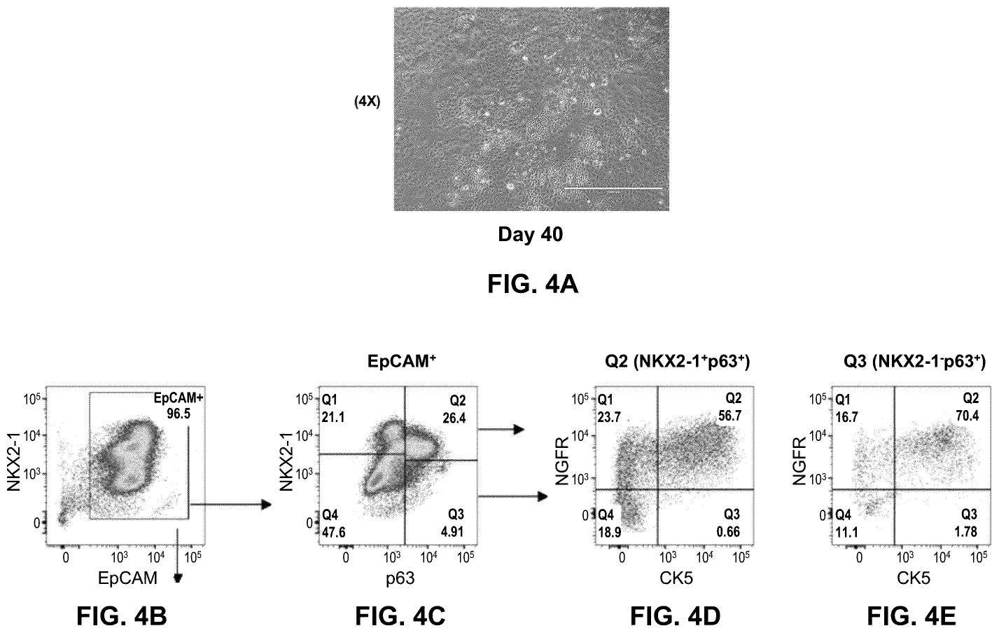

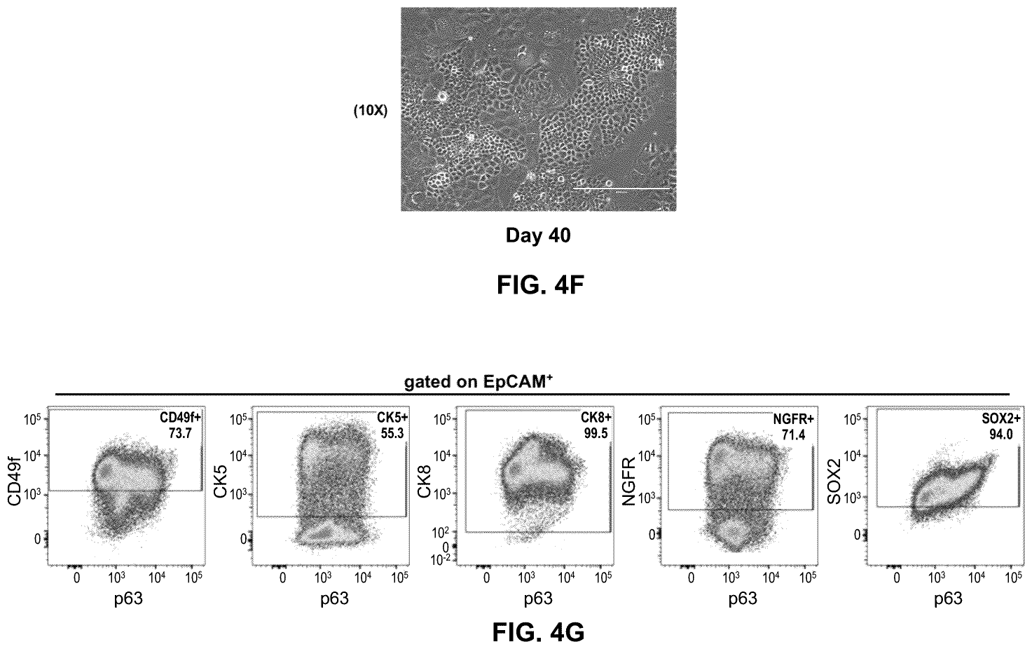

Media was replaced every 24 hours until approximately 95-100% confluency. An exemplary image of cells at Day 40 of differentiation is shown in FIG. 4A (4× magnification) and FIG. 4F (10× magnification). QC plots of cell surface markers EpCAM, NKX2-1, p63, CK5 and NGFR from day 40 are shown in FIG. 4B-4E. QC plots of cell surface markers CD49f, p63, CK8, NGFR and SOX2 from day 40 are shown in FIG. 4G.

### Basal Cell Expansion (Day 41+)

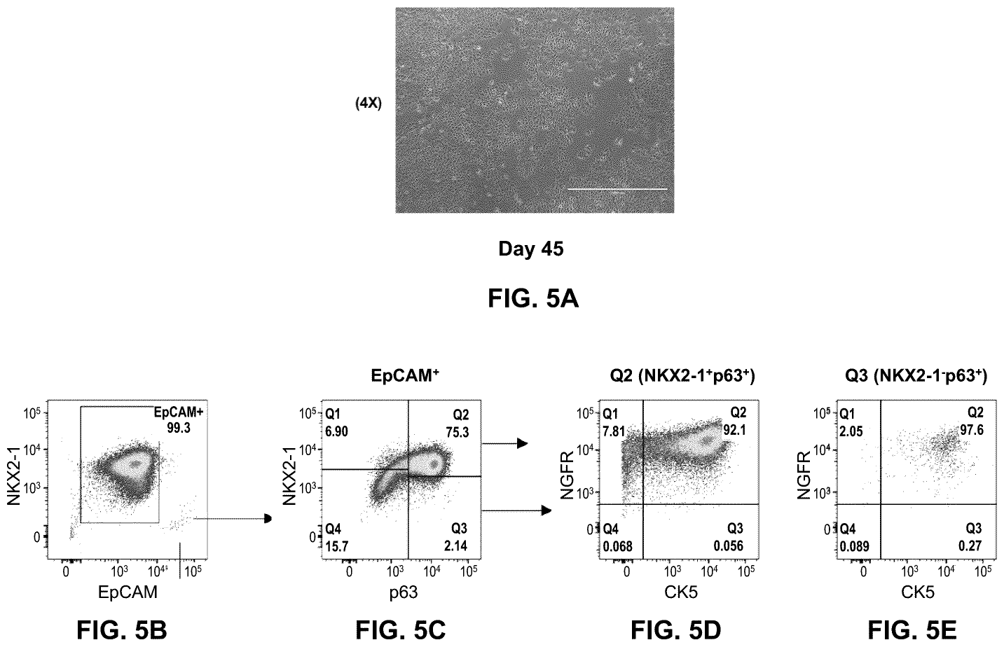

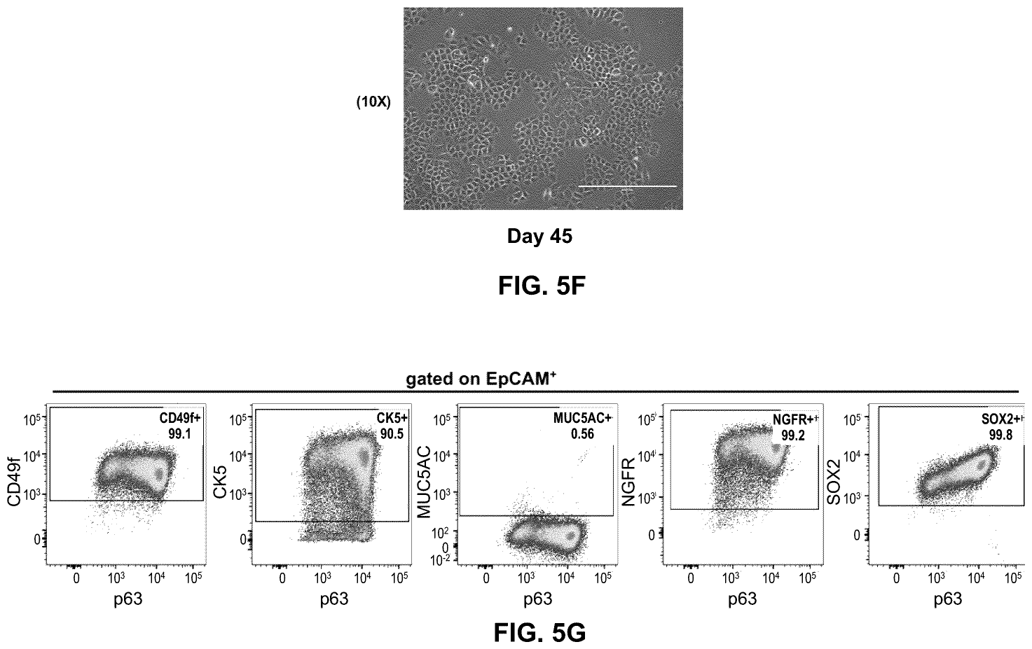

Laminin-521-coated plates were prepared as described above. Spent media was removed from wells and cells were washed with PBS once. Cells were harvested as described above and seeded with Basal Maturation Media on Laminin-521-coated plates at a cell density of approximately 5,000 cells/cm². Cells were then incubated for 24 hours at 37° C. Media was replaced every 24 hours until approximately 95-100% confluency. An exemplary image of cells at Day 45 of differentiation is shown in FIG. 5A (4× magnification) and FIG. 5F (10× magnification). QC plots of cell surface markers EpCAM, NKX2-1, p63, CK5 and NGFR from day 45 are shown in FIG. 5B-5E. QC plots of cell surface markers CD49f, p63, CK8, NGFR and SOX2 from day 45 are shown in FIG. 5G.

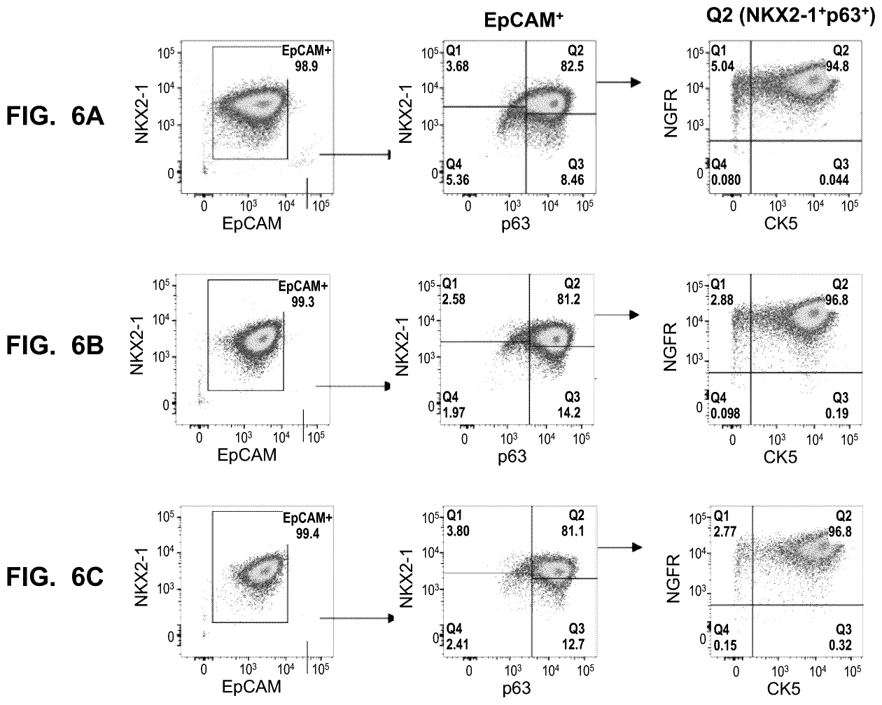

FIG. 6A shows a flow cytometry gating strategy to identify EpCAM⁺, NKX2-1⁺, p63⁺, CK5⁺, and NGFR⁺ iPS-BC cells at day 44 before cryopreservation. FIG. 6B shows a flow cytometry gating strategy to identify EpCAM⁺, NKX2-1⁺, p63⁺, CK5⁺, and NGFR⁺ control iPS-BC cells at day 47. FIG. 6C shows a flow cytometry gating strategy to identify EpCAM⁺, NKX2-1⁺, p63⁺, CK5⁺, and NGFR⁺ iPS-BC cells after thaw from cryopreservation.

All publications, patents and patent applications mentioned in this application are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for differentiating lung progenitor cells (LPC) into epithelial or basal cells, comprising:
(i) culturing LPC in a base culture medium comprising fibroblast growth factor 2 (FGF2), fibroblast growth factor 10 (FGF10), dexamethasone, cyclic adenosine monophosphate (cAMP), an inhibitor of cyclic nucleotide phosphodiesterases, and a Rho-associated, coiled-coil containing protein kinase (ROCK) inhibitor;
(ii) separating the cells of (i) having no or low expression of nerve growth factor receptor (NGFR) to form basal precursor cells;
(iii) culturing the cells of (ii) in a base culture medium comprising FGF10, keratinocyte growth factor (KGF), a transforming growth factor β type I receptor kinase (ALK5) inhibitor, an inhibitor of suppressor of mothers against decapentaplegic (SMAD) phosphorylation, and a ROCK inhibitor; and
(iv) separating the cells of (iii) having expression of NGFR to form epithelial or basal cells.

2. The method of claim 1, further comprising:
(v) culturing the cells of (iv) having expression of NGFR in a base culture medium comprising FGF10, KGF, an ALK5 inhibitor, an inhibitor of SMAD phosphorylation, and a ROCK inhibitor.

3. The method of claim 2, wherein the culturing of (v) is on a laminin-coated surface.

4. The method of claim 3, wherein the laminin is laminin-521.

5. The method of claim 1, wherein the culturing of (i) is in a three dimensional (3D) matrix.

6. The method of claim 1, wherein the culturing of (iii) is in a 3D matrix.

7. (canceled)

8. The method of claim 1, wherein FGF2 is present in the culture medium at a concentration of about 250 ng/mL.

9. The method of claim 1, wherein FGF10 is present in the culture medium of (i) at a concentration of about 100 ng/mL.

10. The method of claim 1, wherein FGF10 is present in the culture medium of (ii) and (iv) (iii) at a concentration of about 20 ng/mL.

11. The method of claim 1, wherein dexamethasone is present in the culture medium at a concentration of about 50 nM.

12. The method of claim 1, wherein cAMP is present in the culture medium at a concentration of about 100 μM.

13. The method of claim 1, wherein the inhibitor of cyclic nucleotide phosphodiesterases is 3-isobutyl-1-methylxanthine (IBMX).

14. The method of claim 13, wherein IBMX is present in the culture medium at a concentration of about 100 μM.

15. The method of claim 1, wherein the ROCK inhibitor is Y-27632.

16. The method of claim 15, wherein Y-27632 is present in the culture medium at a concentration of about 10 μM.

17. The method of claim 1, wherein KGF is present in the culture medium at a concentration of about 20 ng/mL.

18. The method of claim 1, wherein the ALK5 inhibitor is A83-01.

19. The method of claim 18, wherein A83-01 is present in the culture medium at a concentration of about 1 μM.

20. The method of claim 1, wherein the inhibitor of SMAD phosphorylation is DMH-1.

21. The method of claim 20, wherein DMH-1 is present in the culture medium at a concentration of about 0.5 μM.

22. Epithelial cells made by the method of claim 1.

23. Basal cells made by the method of claim 1.

24. An organoid comprising the epithelial cells of claim 22.

25. An organoid comprising the basal cells of claim 23.

26. A differentiation medium comprising a base culture medium, FGF10, KGF, an ALK5 inhibitor, an inhibitor of SMAD phosphorylation, and a ROCK inhibitor.

27. A differentiation medium comprising a base culture medium, about 20 ng/mL FGF10, about 20 ng/mL KGF, about 1 μM A83-01, about 0.5 μM DMH-1, and about 10 μM Y-27632.
